(19) United States
(12) Patent Application Publication
(10) Pub. No.: US 2007/0207211 A1
(43) Pub. Date: Sep. 6, 2007

(54) Emulsion-based microparticles and methods for the production thereof

(75) Inventor: Ehud Zeigerson, Fort Collins, CO (US)

Correspondence Address: THE MCCALLUM LAW FIRM, P. C., 685 BRIGGS STREET, PO BOX 929, ERIE, CO, 80516, US

(73) Assignee: PR Pharmaceuticals, Inc., Fort Collins, CO (US)

(21) Appl. No.: 11/799,700

(22) Filed: May 1, 2007

Related U.S. Application Data
(63) Continuation-in-part of application No. 10/553,003, filed on Aug. 15, 2006.
(60) Provisional application No. 60/461,860, filed on Apr. 10, 2003.

(51) Int. Cl. A61K 9/14; B29C 39/10

(52) U.S. Cl. 424/489; 264/4

(57) ABSTRACT

The apparatus, methods and compositions of the present invention are of use for the production of emulsion-based microparticles containing a biological or chemical agent. The apparatus and methods of the present invention provide for a low-shear, non-turbulent, production of emulsion-based microparticles that provides a narrow, reproducible, particle size distribution, capable of use with both large and small volumes that is conveniently scaled up while providing predictable emulsion properties. The methods include production of emulsion-based microparticles containing a biological or chemical agent which, when suspended in a diluent, are syringable through a needle without clumping of the microparticles or clogging of the needle.

### CROSS REFERENCE TO RELATED APPLICATIONS

This application a Continuation-in-Part of U.S. Ser. No. 10/553,003, filed Oct. 11, 2005, which is a national phase application filed under 35 U.S.C. § 371 of PCT Application Number PCT/US2004/011485, filed Apr. 12, 2004, which claims priority from U.S. Provisional Patent Application No. 60/461,860 filed Apr. 10, 2003.

### FIELD OF THE INVENTION

The invention relates to an apparatus for preparing microparticles, a method of using such apparatus, and a composition made by the method of the invention. More particularly, the disclosed apparatus, methods and composition concern the production of emulsion-based microparticles and a method for producing emulsion-based microparticles containing biological or chemical agents.

### BACKGROUND OF THE INVENTION

Encapsulation of pharmaceuticals in biocompatible, biodegradable polymer microparticles can prolong the maintenance of therapeutic drug levels relative to administration of the drug itself. Sustained release may be extended up to several months depending on the formulation and the active molecule encapsulated. In order to prolong the existence at the target site, the drug may be formulated within a matrix into a slow release formulation (see, for example, Langer (1998)Nature, 392, Supplement, 5-10). Following administration, drug then is released via diffusion out of, or via erosion of the matrix. Encapsulation within biocompatible, biodegradable polyesters, for example, copolymers of lactide and glycolide, has been utilized to deliver small molecule therapeutics ranging from insoluble steroids to small peptides. Presently, there are over a dozen lactide/glycolide polymer formulations in the marketplace, the majority of which are in the form of microparticles.

In addition, U.S. Pat. No. 6,706,289, hereby incorporated in its entirety by reference, discloses controlled release formulations of biologically active molecules that are coupled to hydrophilic polymers such as polyethylene glycol and methods of their production. The formulations are based on solid microparticles formed of the combination of biodegradable polymers such as poly(lactic acid) (PLA), poly(glycolic acid) (PGA), and copolymers thereof.

Several techniques for the production of microparticles containing biological or chemical agents by an emulsion-based manufacturing technique have been reported. In general, the methods have a first phase consisting of an organic solvent, a polymer and a biological or chemical agent dissolved or dispersed in the first solvent. The second phase comprises water and a stabilizer and, optionally, the first solvent. The first and the second phases are emulsified and, after an emulsion is formed, the first solvent is removed from the emulsion, producing hardened microparticles.

An alternative method involves the formation of a “double emulsion”. In this method, a first phase, often called an “internal phase”, is produced and normally consists of water, a biological or chemical agent, and, possibly, a stabilizer. A second-phase normally consists of an organic solvent and a polymer. The first and second phases are emulsified to form a water-in-oil “internal emulsion”. A third-phase usually consists of water, a surfactant and, optionally, the second solvent. The internal emulsion is then emulsified again with the third phase to form an oil-in-water “external emulsion”. After the external emulsion is formed, the organic solvent is removed from the emulsion, producing hardened microparticles.

Emulsions may be formed by a variety of techniques. One such technique is the use of a batch device for mixing the first and second phases under turbulent conditions such as with a stirrer. Other batch processes may employ a homogenizer or a sonicator. In another technique, an emulsion is formed by continuously mixing the first phase and second phase, in-line, using turbulent flow conditions, as in the use of an in-line dynamic mixer or an in-line static mixer.

When emulsions are created by a turbulent mixing device, such as static and dynamic mixers, a turbulent region exists where the two phases mix and the emulsion is formed. This mixing technique is problematic because turbulent mixers create areas of varying turbulence as some areas in the mixer produce a higher turbulence (typically closer to the blades and walls), while other areas produce lower turbulence (further away from blades and walls). Varying turbulence within the mixer results in a wide range of microparticle sizes, which can be undesirable.

Another problem with using turbulent mixing devices for producing microparticles is that a whole range of parameters such as flow rates, viscosities, densities, surface tension and temperature govern the level of turbulence inside the apparatus itself. The sensitivity of a turbulent process to the fluid flow and other physical properties makes it difficult to consistently produce a final product with the same properties. Batch to batch variation is not acceptable for the majority of microparticle products.

Another problem with turbulent mixing processes for the production of microparticles is that some active agents, such as proteins, are sensitive to high shear forces that are inherently part of turbulent mixing. Hence, these processes cannot be utilized to create microparticles with some common biological or chemical agents.

An additional difficulty with turbulent mixing processes relates to scalability. Turbulence, and the resulting microparticle properties, cannot be accurately predicted when changing the scale of production. This means that any time a change is made to the turbulent emulsion apparatus, a new set of experiments must be conducted in order to establish new guidelines for operation of the device in order to create the desired microparticle product. The need for repeated testing whenever scaling up production is expensive and time-consuming.

Turbulent-based emulsion devices also have physical limitations, specifically with their application of the laws of fluid dynamics. When using turbulent flow mixing devices, the dynamics of a particular mixer is correlated with a particular microparticle size and microparticle size distribution.

In order to achieve the same microparticle size and distribution when scaling up or scaling down, the same mixing turbulence must be produced in the larger or smaller mixer. As scaling up involves a change in the size of the mixer, a change in velocity (V) must be accomplished in order to compensate for the change in the diameter (D) of the mixer. Thus, application of a turbulent-based process for the formation of microparticles becomes especially difficult, and ultimately not practical, when very low flow rates are desirable because it is hard to achieve the desirable turbulence.

In the above-mentioned production processes, the resultant particle size is a function of the shear forces experienced by the two phases when mixed. Shear forces in these methods vary across the volume being mixed and, as a result, produce relatively broad particle size distributions. In the case of production processes dependent on turbulent flow, it is difficult to achieve turbulent flow conditions for low flow rates such as might be used during exploratory experiments with limited volumes, and the performance of larger devices is difficult to predict from results with small versions. Hence, there is little correlation between the results achieved on a small-scale in the laboratory and those achieved in later manufacture-sized production with turbulent flow based production processes.

An alternative method for producing an emulsion utilizes a packed bed emulsifier, as described in U.S. Pat. No. 4,183,681 ('681). The '681 patent describes the use of a packed bed emulsifier to form oil-in-water emulsions. Unfortunately, the emulsions disclosed do not form microparticles, are directed to applications with oil/water phase volume ratios equal to or greater than 1:1 and the packing materials that are found to be effective are not compatible with the need for clean and sterile apparatus such as required for microparticles containing therapeutic chemical or biological agents.

Other alternative emulsion forming techniques may employ filtration membranes or passage of fluids through a microchannel device as described in U.S. Pat. No. 6,281,254. These methods require precision fabrication and can be cumbersome to scale up to production volumes.

Another problem with turbulent mixing techniques used in the formation of microparticles is that such microparticles are difficult to inject through narrow gauge needles without clogging or clumping, thus requiring larger gauges when administering them via a needle. Generally, biodegradable polymer-based controlled delivery products, manufactured using turbulent mixing techniques and now on the market, use relatively large gauge needles, typically from 18-22 gauge, for injection of microparticles suspended in a fluid medium, typically aqueous. Patient comfort and therefore compliance with therapy would be greatly aided by use of smaller needles such as 25 gauge or smaller, for example 27, 29 and 30 gauge.

Thus, a method is needed for forming emulsion-based microparticles that provides a narrow, reproducible, particle size distribution, capable of use with both large and small volumes, and is capable of being conveniently scaled up while providing predictable emulsion properties. Ideally, this method would utilize a non-turbulent emulsifier in order to allow its use with all chemical or biological agents.

### SUMMARY OF THE INVENTION

In contrast to known methods of producing microparticles dependent on turbulent flow, such as that created with a static or dynamic mixer, the apparatus and methods of the invention utilize laminar flow conditions to produce an emulsion that results in a composition comprising microparticles containing biological or chemical agents after solvent removal.

In one embodiment the invention is directed to a composition comprising a plurality of microparticles prepared by forming a first phase, the first phase comprising a solvent, active agent and a polymer; forming a second phase comprising a solvent; passing the first phase and the second phase through a packed bed apparatus under laminar flow conditions, and collecting the microparticles containing the active agent; wherein the microparticles, suspended in a diluent, are syringeable through a 25-gauge or narrower needle.

In another embodiment, the invention is directed to a method of preparing microparticles, comprising forming a first phase, said first phase comprising a solvent, active agent and a polymer; forming a second phase comprising a solvent; passing said first phase and said second phase through a packed bed apparatus under laminar flow conditions, wherein said method results in the formation of microparticles; and collecting said microparticles containing said active agent; wherein said microparticles, suspended in a diluent, are syringeable through a needle.

In another embodiment, the invention is directed to a method of preparing microparticles comprising forming a first phase comprising an organic solvent; forming a second phase comprising an aqueous solvent, one or more active agents and a polymer; passing said first phase and said second phase through a packed bed apparatus under laminar flow conditions, resulting in the formation of microparticles; and collecting said microparticles containing an active agent; wherein said microparticles, suspended in a diluent, are syringable through a needle.

In yet another embodiment, the invention is directed to a method for preparing microparticles, comprising forming a first phase comprising an organic solvent; forming a second phase comprising an organic solvent, one or more active agents and a polymer; passing said first and second phases through a packed bed apparatus under laminar flow conditions, resulting in the formation of microparticles; and collecting said microparticles containing an active agent; wherein said microparticles, suspended in a diluent, are syringable through a needle.

In a still further embodiment, the invention is directed to a method for preparing microparticles, comprising forming a first phase comprising an aqueous solvent, one or more active agents and an emulsion stabilizer; forming a second phase comprising an organic solvent and a polymer; forming a third phase comprising an aqueous solvent; passing said first and second phases through a packed bed apparatus to form a first emulsion; passing said first emulsion and said third phase through a second packed bed apparatus, resulting in the formation of microparticles; collecting said microparticles containing said active agent; wherein said microparticles, suspended in a diluent, are syringable through a needle.

In a further embodiment, the invention is directed to an apparatus for the preparation of emulsion-based syringable microparticles containing biological or chemical agents, wherein the apparatus comprises a vessel and packing material situated therein.

### BRIEF DESCRIPTION OF THE DRAWINGS

The following drawings form part of the specification and are included to further demonstrate certain embodiments. These embodiments may be better understood by reference to one or more of these drawings in combination with the detailed description of specific embodiments presented herein.

FIG. 1. illustrates an exemplary packed bed apparatus with various components according to an embodiment of the invention.

FIG. 2. illustrates an exemplary emulsion system for manufacturing microparticles containing a biological or chemical agent including a packed bed apparatus according to an embodiment of the invention.

### DETAILED DESCRIPTION OF THE INVENTION

Definitions

For purposes of the invention, the following terms shall have the following meanings:

The term “a” or “an” entity refers to one or more of that entity; for example, “a protein” or “an estradiol metabolite molecule” refers to one or more of those compounds or at least one compound. As such, the terms “a” or “an”, “one or more” and “at least one” can be used interchangeably herein. It is also to be noted that the terms “comprising,” “including,” and “having” can be used interchangeably. Furthermore, a compound “chosen from one or more of” refers to one or more of the compounds in the list that follows, including mixtures (i.e. combinations) of two or more of the compounds.

The term “biodegradable” refers to polymers that dissolve or degrade in vivo within a period of time that is acceptable in a particular therapeutic situation. This time is typically less than five years and usually less than one year after exposure to a physiological pH and temperature, such as a pH ranging from 6 to 9 and a temperature ranging from 25 C to 38 C.

The term “packed bed apparatus” refers to any vessel containing packing material capable of creating an emulsion upon contact with two immiscible fluids.

The term “active agent” refers to any biological or chemical agent.

The term “microparticles” refers to particles having a diameter of typically less than 1.0 mm, and more typically between 1.0 and 250 microns. The microparticles of the present invention include, but are not limited to, microspheres, microcapsules, microsponges, microgranules and particles in general, with an internal structure comprising a matrix of agent and excipient. Microparticles may also include nanoparticles.

The term “nanoparticles” refers to particles having a diameter of typically between about 20 nanometers (nm) and about 2.0 microns (μm), more typically between about 100 nm and about 1.0 μm.

An “injection” is a preparation intended for parenteral administration. Injections include, but are not limited to, liquid preparations that are drug substances or solutions or suspensions thereof.

The term “controlled release” refers to control of the rate and/or quantity of biologically active molecules delivered according to the drug delivery formulations of the invention. The controlled release kinetics can be continuous, discontinuous, variable, linear or non-linear. This can be accomplished using one or more types of polymer compositions, drug loadings, inclusion of excipients or degradation enhancers, or other modifiers, administered alone, in combination or sequentially to produce the desired effect. “Controlled release” microparticles include, but are not limited to, “sustained release” microparticles and “delayed release” microparticles.

The term “sustained release” refers to releasing a biologically active agent into the body steadily, over an extended period of time. Sustained release formulations offer the ability to provide a subject with a biologically active agent over a time period greater than that achieved by a typical bolus administration of the biologically active agent. Sustained release microparticles may advantageously reduce the dosing frequency of a biologically active agent.

A “biologically active agent”, “bioactive agent” “biologically active moiety” or “biologically active molecule” can be any substance which can affect any physical or biochemical properties of a biological organism, including but not limited to, viruses, bacteria, fungi, plants, animals, and humans. Biologically active molecules can include any substance intended for diagnosis, cure mitigation, treatment, or prevention of disease in humans or other animals, or to otherwise enhance physical or mental well being of humans or animals.

By “treating” is meant the medical management of a patient with the intent that a cure, amelioration, stasis or prevention of a disease, pathological condition, or disorder will result. This term includes active treatment, that is, treatment directed specifically toward improvement of a disease, pathological condition, or disorder, and also includes causal treatment, that is, treatment directed toward removal of the cause of the disease, pathological condition, or disorder. In addition, this term includes palliative treatment, that is, treatment designed for the relief of symptoms rather than the curing of the disease, pathological condition, or disorder; preventive treatment, that is, treatment directed to prevention of the disease, pathological condition, or disorder; and supportive treatment, that is, treatment employed to supplement another specific therapy directed toward the improvement of the disease, pathological condition, or disorder. The term “treating” also includes symptomatic treatment, that is, treatment directed toward constitutional symptoms of the disease, pathological condition, or disorder.

The term “syringability” refers to uptake and delivery of microparticles through a needle without substantial clumping of the particles or clogging of the needle.

The invention provides an apparatus, methods of using such apparatus for the production of microparticles via an emulsion-based technique, and a composition made by the method of the invention. In contrast to previously known methods for the production of emulsion-based microparticles, the invention provides a non-turbulent, or laminar flow, process for producing microparticles with a narrow, reproducible, particle size distribution, capable of use with both large and small volumes with the capacity of being scaled up, while providing consistent predictable properties in the resulting larger batches. Microparticles containing many biological or chemical agents may be produced by the methods of the invention.

The invention overcomes disadvantages of previous methods of microparticle production through the use of a non-turbulent or laminar flow, packed bed system rather than a mixer. Both static and dynamic mixers create turbulent flow conditions associated with highly variable microparticle size distributions. The use of a packed bed system to create an emulsion provides for uniform droplets and resultant microparticle size distribution, as well as conditions suitable for many chemical or biological agents. Additionally, the apparatus and methods of the invention can easily produce scalable results. Desirable batches of microparticles produced in the laboratory on a small scale can easily be reproduced on a larger manufacturing scale merely by utilizing the same packing material in a vessel with a larger diameter. This allows for the inexpensive and efficient scaling of the production process once the desired microparticles are produced on a small scale in the laboratory.

In a certain embodiment, the methods of the invention provide a continuous process for making an emulsion for microparticle production in a wide range of flow rates and volumes. In some embodiments, the methods involve a process for making microparticles with a pre-determined size distribution. In alternative embodiments, the methods provide a continuous process for making microparticles at very low flow rates.

Microparticles of the invention may be made by any suitable emulsion technique known in the art. In one embodiment, the method for producing an emulsion for microparticle production includes (1) forming a first phase typically containing an organic solvent, a polymer, and one or more biologically active agents and/or chemicals; (2) forming a second phase typically containing water as the second solvent, an emulsion stabilizer and optionally a solvent; and (3) passing the first and second phases through a packed bed apparatus to form an “oil in water” type emulsion.

In another embodiment, the method for production of an emulsion includes (1) forming a first phase typically containing an organic solvent and an emulsion stabilizer; (2) forming a second phase typically containing water as the second solvent, one or more biologically active agents and/or chemicals, and a water soluble polymer; and (3) passing the first and the second phases through a packed bed apparatus to form a “water in oil” type emulsion.

In a third embodiment, the invention provides methods for producing emulsions by (1) forming a first phase containing an organic solvent and, optionally, an emulsion stabilizer; (2) forming a second phase containing a second organic solvent, one or more biologically active agents and/or chemicals, and a polymer; and (3) passing the first and the second phases through a packed bed apparatus to form an organic emulsion.

In yet another embodiment the invention provides methods for producing emulsions by (1) forming a first phase typically containing water, one or more biologically active agents and/or chemicals and an emulsion stabilizer; (2) forming a second phase typically containing an organic solvent and a polymer; (3) forming a third phase typically containing water and optionally containing a stabilizer; (4) passing the first and the second phases through a packed bed apparatus to form a “water in oil” type emulsion; and (5) passing the first emulsion and the third phase through a second packed bed apparatus to form a “water in oil in water” emulsion.

The apparatus and methods of using such apparatus to produce microparticles are not dependent on turbulent flow. The methods of making microparticles of the present invention work at laminar flow rates in contrast with prior methods of making microparticles. In the present invention, microparticles with a narrow and repeatedly precise particle size distribution can be produced. Additionally, they can be produced on a small scale and easily scaled-up to manufacturing size by merely altering the diameter of the vessel. This was not possible with prior turbulent flow methodologies. Surprisingly, making the emulsion within a laminar flow regimen solves many of the problems associated with turbulent emulsion-forming processes, as described above.

Packed Bed Apparatus

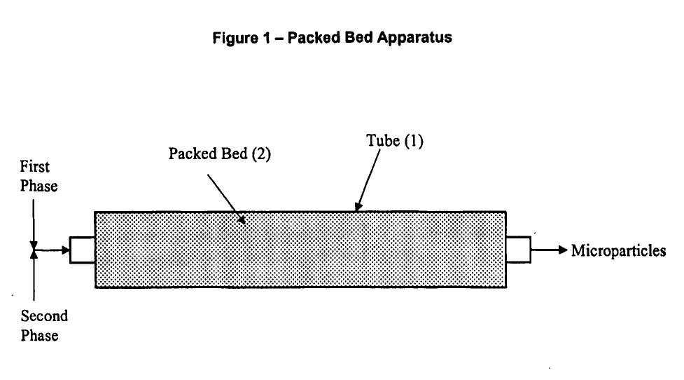

The apparatus of the invention is a packed bed apparatus for the production of microparticles through an emulsion-based technique. Such apparatus may be a vessel of any shape capable of being filled with packing material that allows liquid to flow through it (See FIG. 1). The apparatus of the invention may further provide a material capable of insertion into both ends for enclosure of materials in such apparatus. FIG. 1 illustrates an exemplary apparatus according to one embodiment of the invention. In this embodiment, a tube (1) is filled with beads as packing material (2).

The apparatus of the invention is packed with materials that force the liquids to flow through the gaps in between the packing material in order to pass through the apparatus. The gaps in between the packing material inside the device may be viewed as many channels which cross each other's path repeatedly as the fluids flow through the bed.

In the invention, the emulsion is made as the two fluids, or phases (typically oil and water), are flowing through the gaps inside the packing. As the two phases are flowing through the bed of solids, they cross each other's path repeatedly, and the continuous phase (usually the water) is dividing the discontinuous phase (usually the oil) into droplets, thus creating an emulsion. The discontinuous phase droplet size is being reduced repeatedly until a final droplet size is achieved. Once the discontinuous droplets have reached a certain size, they will not be reduced any further even if they continue flowing through the packing. This emulsion making mechanism allows the formation of a precisely sized emulsion at laminar flow conditions.

The very unique dynamics of a packed bed allow for the production of microparticles continuously at very low flow rates, not possible with mixing devices. This low flow rate enables the consistent production of high-quality microparticles in batches as small as 0.1 grams that maintain consistent particle size distribution. Additionally, these very unique flow dynamics also provide for scalability from laboratory to manufacturing sized batches.

The apparatus and methods of using such apparatus provide an emulsion-based process for making microparticles that is insensitive to flow rates within the laminar flow region. Unlike turbulent mixer-based processes, the methods of the invention are not sensitive to changes in the flow rates, when operated within a laminar flow region. The flow rate of use in the invention can be any laminar flow rate. In one embodiment, the flow rate is 0.0001 to 100 liter/minute.

The apparatus and methods of using such apparatus provide an emulsion-based process for making microparticles that is easily scalable from laboratory to manufacturing sized batches. A typical batch may demonstrate 10,000-fold scalability. In a particular batch, the size of the batch may be chosen from one or more of, but not limited to, 0.1 gram, 1 gram, 10 grams, 50 grams, 100 grams, 250 grams, 0.5 kilograms, 1 kilogram, 2 kilogram, 5 kilograms, 10 kilograms, 15 kilograms, 20 kilograms, 25 kilograms, 30 kilograms, and the like. One method of increasing the scale of a batch of microparticles is to increase the diameter of the vessel. Such increase will function to increase the volume of emulsion through the vessel, thus directly increasing the size of the batch produced.

The apparatus and methods of using such apparatus provide an emulsion-based process for making microparticles that provides for tight control of the particle size distribution. Microparticle size distribution may be manipulated by altering the packing material size, shape and type; rearranging the inlet or outlet enclosures; alteration of the physical properties of the first, second or third phases; altering the length or width of the vessel and the like. For example, the final microparticle size can be determined by the size of the packing material, such as the diameter of a glass bead. Additionally, the length of the vessel may directly affect the particle size distribution.

The vessel of the invention may be in any form capable of containing the packing material. In one embodiment, the apparatus is in the form of a tube. The cross section may be of any compatible shape including rectangular, square and round. In one embodiment, the cross section is approximately circular. The vessel may be of any length. In one embodiment, the length of the vessel may range from 1 cm to 100 meters. In another embodiment, such vessel is 10 to 50 cm in length.

Packing material of use in the invention may be anything capable of inclusion within the device. In one embodiment, such packing material may include, but is not limited to, spheres, beads, pellets, chips, fibers, sponges, pillows, and the like in any shape or form. In one embodiment, the packing material is approximately spherical. Material for the packing may be metal, ceramic, plastic, glass and the like. In one embodiment, the packing material is glass or a non-reactive metal such as stainless steel. In one embodiment, the packing material is boro-silicate glass beads or stainless steel beads. The diameter of the beads may range from 20 to 2000 microns. In one embodiment, the beads may be in the range of 50 to 1000 microns.

The phases may be introduced into the packed bed emulsifier by any method. In one embodiment, the phases are introduced through pipes or tubes and may be pumped, forced by gas or another type of pressure source, fed by gravity or pulled by a vacuum at the discharge side of the packed bed emulsifier. The liquid phases may be carried by pipes comprising stainless steel, glass or plastic compatible with the solvents and temperatures used. The fluid phases may be at ambient temperature or at any temperature required between approximately freezing and approximately boiling for the particular fluid. The apparatus and methods of the invention may be utilized at any pressure compatible with the equipment utilized. The pressure may be adjusted to a pressure necessary to overcome the resistance of the packing bed and provide a flow rate in the laminar flow region.

Microparticles containing a biological or chemical agent are collected from the emulsion product of the packed bed apparatus via solvent extraction. Such techniques are known in the art. Solvent extraction can be done by, but is not limited to, the methods of spray drying, extraction into a water or other liquid bath, freeze-drying, evaporation and the like.

The first and second phases of the present invention are any two fluids that are immiscible with one another. If a third phase is utilized in the production of microparticles, the resulting product from the first and second phases is combined with the third phase. In this case, the product from the combination of the first and second phases and the third phase are any two fluids that are immiscible with one another.

Solvents for the first phase may be any organic or aqueous solvents. Examples of solvents include, but are not limited to, water, methylene chloride, chloroform, ethyl acetate, benzyl alcohol, diethyl carbonate, methyl ethyl ketone and mixtures of the above. In a particular embodiment, the solvent is ethyl acetate or methylene chloride.

The first phase may comprise a solution of a biodegradable polymer and a biological or chemical agent as a solution or suspension. Alternatively the biological or chemical agent is dissolved or suspended in the second phase.

Solvents for the second phase may be any organic or aqueous fluid that is immiscible with the first phase. Examples include, but are not limited to, water, a water-based solution, an organic solvent, and the like. In one embodiment, the second phase contains water, an emulsion stabilizer and optionally a solvent. In another embodiment, the second phase contains water, one or more biological or chemical agents and optionally a water-soluble polymer. In another embodiment, the second phase contains a second organic solvent, one or more biological or chemical agents and a polymer.

Solvents for the third phase solvent may be any organic solvent or water.

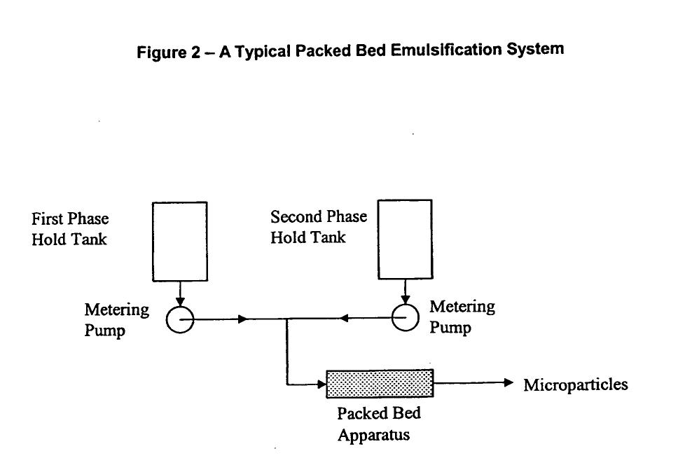

A holding tank or feed vessel may be utilized in the invention to hold the first or second phases (See FIG. 2). The holding tanks or feed vessels may be jacketed or otherwise equipped to provide temperature control of the contents. A tube may run from each one through a pump and later merge with the tube from the other one to the entrance to the packed bed apparatus. The merge may also happen at the entrance of the packed bed apparatus or inside the packed bed apparatus itself. Additionally, the packed bed apparatus may include pumps or other means of moving the phases into and through the packed bed apparatus. The phases may flow from the holding tanks or feed vessels into the packed bed apparatus without pumps, by simple gravity, by pressure or by a vacuum from the other end of the packed bed apparatus, and the like. The tubes may further include addition of flow meters, feedback control, flow rate programming via programmed logic control, and the like.

Emulsion stabilizers of use in the invention may include, but are not limited to, poly(vinyl alcohol), polysorbate, protein such as albumin, poly(vinyl pyrrolidone). In a particular embodiment, poly(vinyl alcohol) is used. The concentration of emulsifier may be in the range 0% to 20%, and in one embodiment, 0.5% to 5%. Emulsion stabilizers may be included in one or both of the first and second phases.

The polymer of the invention may be any polymer. In one embodiment, the polymer comprises a biodegradable polymer. Biodegradable polymers of use in the invention include, but are not limited to, poly(d,l-lactic acid), poly(l-lactic acid), poly(glycolic acid), copolymers of the foregoing including poly(d,l-lactide-co-glycolide) (PLGA), poly(caprolactone), poly(orthoesters), poly(acetals), poly(hydroxybutryate). In one embodiment, the biodegradable polymer is PLGA. PLGA may have a monomer ratio of lactide:glycolide in the range of about 40:60 to 100:0 or from about 45:55 to 100:0. In another embodiment, the polymer comprises block copolymers of hydrophilic and hydrophobic polymers.

In one embodiment, the inherent viscosity of the biodegradable polymer may be in the range 0.1 to 2.0 dL/g. In another embodiment, the range is from about 0.1 to about 1.0 dL/g. The biodegradable polymer is included at a concentration in the range 1% to 40% w/w, and in one embodiment, in the range 5%-20% w/w.

Active agents of the invention may be any biological or chemical agent. Examples of biologically active agents include, but are not limited to, antibodies, peptides, proteins, enzymes, fusion proteins, porphyrins, nucleic acids, nucleosides, oligonucleotides, antisense oligonucleotides, RNA, DNA, siRNA, RNAi, aptamers, and small molecule drugs. Other biologically active agents include, but are not limited to, dyes, lipids, cells, and viruses. Biological agents of use in the invention may be any agent capable of having an effect when administered to an animal or human. In one embodiment, they include, but are not limited to, an organic molecule, an inorganic molecule, antiinfectives, cytotoxics, antihypertensives, antifungal agents, anti-anxiety agents, anti-inflammatory agents, anti-tumor agents, anti-tubulin agents, antipsychotics, antibodies, proteins, peptides, antidiabetic agents, immune stimulants, immune suppressants, antibiotics, antivirals, anticonvulsants, antihistamines, cardiovascular agents, anticoagulants, hormones, antimalarials, analgesics, anesthetics, nucleic acids, steroids, aptamers, blood clotting factors, hemopoietic factors, cytokines, interleukins, colony stimulating factors, growth factors, growth factor analogs, fragments thereof and the like. In another embodiment biological agents include PEGylated bioactive agents. In another embodiment, a biologically active molecule is conjugated to a non-toxic, long-chain, hydrophilic, hydrophobic or amphiphilic polymer. In a further embodiment, a bioactive agent such as insulin is conjugated to polyethylene glycol.

Chemical agents of use in the invention may be any synthetic or natural agent. In a particular embodiment, they include, but are not limited to, antioxidants, porosity enhancers, solvents, salts, cosmetics, food additives, textile-chemicals, agro-chemicals, plasticizers, stabilizers, pigments, opacifiers, adhesives, pesticides, fragrances, antifoulants, dyes, oils, inks, catalysts, detergents, curing agents, flavors, foods, fuels, herbicides, metals, paints, photographic agents, biocides, pigments, plasticizers, propellants, solvents, stabilizers, polymer additives and the like.

The methods of the invention are functional at any temperature within the operating range of the equipment, solvents and active agent. Factors that determine the appropriate temperature for a particular process include the optimum temperature for the two phases to be pumped through the packed bed apparatus. If a third phase is utilized, the temperature for the first packed bed apparatus may be the same or different than that of the second packed bed apparatus. The temperature needs to be such that the two phases are of a desirable viscosity. Additionally, the solubility of the polymer and active molecule may require an increase in temperature in order to produce a complete solution. The temperature may additionally be affected by the stability limit of the biological or chemical agent. Typical operating temperatures may range in one embodiment, from 18 to 22° C., in one embodiment from 15 to 30° C., in another embodiment from 10 to 70° C., in another embodiment from 0 to 96° C., and the like. In general, temperature may range from −273 to 150° C.

The microparticles of the invention can be used for any purpose. In one embodiment, they are administered to a patient. They may be administered to patients in single or multiple doses. The microparticles may also be administered in a single dose form that functions to further release the biological or chemical agent over a prolonged period of time, eliminating the need for multiple administrations.

The microparticles of the invention can be stored as a dry material. In the instance of administration to a patient, prior to such use, the dry microparticles can be suspended in an injection vehicle. Upon suspension, the microparticles may then be injected into the patient or otherwise utilized.

Injection vehicle (“diluent”, “injection medium”, “injection solution”, “pharmaceutical liquid vehicle”, “suspension medium”, “excipient”, “carrier”) is an aqueous or non-aqueous liquid for suspending and injecting microparticles. Aqueous injection vehicles comprise water and any or all of buffer, salts, non-ionic tonicity compounds, viscosity enhancers, stabilizers, antimicrobials, and surfactants. In one embodiment, the microparticles are suspended in an injection solution comprising SDS, Tween 20 or mannitol. In one embodiment the injection vehicle is 0.5%-2.5% sodium carboxymethylcellulose in water. In another embodiment the injection vehicle is 0-1.5% (w/w) sodium carboxymethylcellulose, 0-0.5% (w/w) Tween-80 or Tween-20, 0-330 mM NaCl, 0-10 mM sodium phosphate in water, pH 5-9. In one embodiment, the microparticles are suspended in an injection solution comprising 0.5% SDS. In a further embodiment the injection vehicle comprises 0.2% Tween-20 in water.

Microparticles can vary in size, ranging from submicron to millimeter diameters. Microparticle size is partially determined by the size and shape of individual packing material particles of the packed bed emulsifier. Large and misfit packing materials generally pack together less closely than smaller packing material particles and produce larger gaps for the fluids to flow through. Larger gaps in the packing material produce larger microparticles and smaller gaps in the packing material produce smaller microparticles. The flow rate does not affect the size of the microparticles produced from a particular apparatus. In one embodiment, microparticles are 1-200 microns in order to facilitate administration to a patient through a syringe needle. In a particular embodiment, the microparticles are between 10-100 microns. In another embodiment the microparticles are less than 63 microns. In another embodiment the microparticles are between 25-45 microns. In still another embodiment the microparticles are in the range 25-63 microns. In another embodiment the microparticles have a mean diameter less than 45 microns. In another embodiment the microparticles have a mean diameter between about 35 microns and about 45 microns. In another embodiment the microparticles have a mean diameter between about 25 microns and about 35 microns. In one embodiment the microparticles have a diameter of less than or equal to 75% of the inner diameter of the needle. In another embodiment the microparticles have a diameter of less than or equal to 50% of the inner diameter of the needle. In a further embodiment, the diameter is less than or equal to 35% of the inner diameter of the needle. In yet another embodiment, the microparticle diameter is less than or equal to 25% of the inner diameter of the needle. In a still further embodiment, the microparticle diameter is less than or equal to 10% of the inner diameter of the needle.

For medical applications, the diameters of the microparticles range from about 1 micron (μm) to about 200 μm. In another embodiment, the microparticles have a particle size distribution ranging from about 1 μm to about 100 μm in diameter. In another embodiment, the microparticles have a particle size distribution ranging from about 10 μm to about 50 μm in diameter. Microparticle size distribution may be measured by several methods including, but not limited to, laser light diffraction, scanning electron microscopy, visible light microscopy and electrical sensing zone method. The results may be expressed as an average (mean or mode) value, a standard deviation or half width, the diameters below which 10%, 50% and 90% of the particles are found (d₁₀, d₅₀, d₉₀), and the fraction of the microparticles within a given range, among others. Further, data may be expressed by volume weighted or number weighted statistics. For the instant description, laser diffraction measurements are used, volume weighted statistics are employed, the average is expressed as a mean value, both d₁₀, d₅₀, d₉₀ etc. and fraction within a range are used to describe particle size distributions.

The use of a packed bed emulsifier to form microparticles provides a narrow size distribution centered at a desired mean diameter with most of the particles contained in a desired range. During the final steps of microparticle manufacturing, filtration is typically used to exclude particles with diameters lower or higher than the desired cutoffs. With traditional microparticle manufacturing involving turbulent mixing, the particle size distribution is broad, and the yield in a narrow range may be too low to be economical so that a wide particle size range is necessitated. With a packed bed emulsifier of the present invention, operated as described, narrow particle size distributions are obtained, and final filtration (sieving) to achieve the desired cutoff diameters produces a high yield of microparticles. Without being bound by theory, the narrow particle size distribution obtained in the present invention may contribute to the capability for the microparticles of the present invention to be syringed through small needles.

In one embodiment the microparticles are predominantly in the range about 1 um to about 6 um. In another embodiment the microparticles are in the range about 10 um to about 25 um. In still another embodiment the microparticles are in the range about 25 um to about 80 um.

In another embodiment, the microparticles have an average diameter of less than about 45 μm. In another embodiment, the microparticles have an average diameter of about 30 μm. In a further embodiment the microparticles are predominantly in the range 10 to 30 um. In another embodiment >80% of the microparticles have diameters in the range 25-63 μm. In another embodiment >90% of the microparticles have diameters in the range 25-63 μm.

In one embodiment, the microparticles are of a suitable size and morphology allowing for delivery though a needle having a small inner-diameter, such as a 25 gauge needle or narrower. Microparticles are referred to herein as “syringable” if they are able to be taken up and delivered though a needle without substantial clumping of the microparticles or clogging of the needle. In one embodiment the microparticles are syringable through a 25 gauge needle or narrower. Microparticles are referred to herein as “injectable” if they consistently can be injected into a desired locus of a subject though a needle. In one embodiment, the microparticles are injectable through a 25 gauge needle or narrower.

In one embodiment particularly suited for medical applications, the microparticles are syringable through a needle having a gauge of at least 25 and having a nominal inner diameter of 0.0095 inches (241 microns) or less. In another embodiment, the microparticles are syringable through a needle having a gauge of at least 27 and having a nominal inner diameter of 0.0075 inches (190 microns) or less. In another particular embodiment the microparticles are syringable through a needle having a gauge of at least 29 and having a nominal inner diameter of 0.0065 inches (165 microns) or less. In another particular embodiment the microparticles are syringable through a needle having a gauge of at least 30 and having a nominal inner diameter of 0.0055 (140 microns) inches or less. In one embodiment, when suspended in an injection vehicle at a concentration of 50-600 mg/ml, the microparticles are syringable through a needle having a gauge of 25, 27, 29 or 30. In one embodiment, when suspended in an injection vehicle at a concentration of 50-200 mg/ml, the microparticles are syringable through a needle having a gauge of 25, 27, 29 or 30. In one embodiment, when the microparticles are suspended in an injection vehicle at a concentration of 100 to 600 mg/ml, the microparticles are syringable through a needle having a gauge of 25 or 27.

In one embodiment, the microparticles flow freely without the formation of aggregates and are readily suspended in an injection vehicle for injection. Free-flowing and/or un-agglomerated powders are advantageous because they roll with substantially no friction and can be easily placed in containers, suspended or incorporated into a solution suitable for injection. Flowability of microparticles can be measured by any suitable means such as a Jenike Shear Tester (Jenike & Johanson, Inc., Westford, Mass.), which measures the direct shear strength of powders and other bulk solid materials. Using a Jenike Shear Tester, a shear cell (base and ring) is filled with material; a vertical load is applied to the covered cell, using weights and the weight carrier; and the shear cell ring is pushed horizontally across the base, with the required force measured and recorded. Other apparatuses for measuring flowability include a powder rheometer (Freeman Technology, Worcestershire, UK) that measures the force of a twisted blade along a helical path through a powder sample establishing a required flow rate and pattern of flow. A critical orifice and an angle of repose using an avalanche process may also be measured.

The microparticles of the invention have a core load sufficient to deliver a biologically active agent to maintain therapeutically effective levels of the biologically active agent for sustained periods. In one embodiment, the microparticles have a core load of greater than or equal to about 5% by weight of the biologically active agent. In one embodiment, the microparticles have a core load of greater than or equal to about 10% by weight of the biologically active agent. In one embodiment, the microparticles have a core load of greater than or equal to about 15% by weight of the biologically active agent. In another embodiment the microparticles have a core load of greater than or equal to about 20% by weight of the biologically active agent. In another embodiment the microparticles have a core load of greater than or equal to about 30% by weight of the biologically active agent.

In one embodiment, the microparticles release a biologically active agent over a period of at least about 1-12 months. In a further embodiment, the microparticles release a bioactive agent over a period of at least about 3-6 months. In another embodiment the microparticles release a bioactive agent over a period of 1 week to 3 months.

It is well known in the art that the biodegradable polymer composition can be varied to affect the sustained release duration. For example PLGA with a 45:55 lactide:glycolide ratio and an inherent viscosity of 0.15 dL/g releases drug over a one to two week period. The high glycolide content and low molecular weight, reflected by the relatively low inherent viscosity value, lead to rapid hydrolysis of the polyester chains with consequent drug release. On the other hand 85:15 lactide:glycolide PLGA with an inherent viscosity of 0.91, reflecting a molecular weight of over 100,000 daltons gives a much longer drug release profile that can last more than 6 months.

In one embodiment, the microparticles of the invention have high encapsulation efficiency. In one embodiment, the microparticles have an encapsulation efficiency of greater than or equal to about 80%. In another embodiment the microparticles have an encapsulation efficiency of greater than or equal to about 90%. In another embodiment the microparticles have an encapsulation efficiency of greater than or equal to about 95%. In another embodiment the microparticles have an encapsulation efficiency of about 100%.

In one embodiment, the microparticles of the invention have any suitable morphology. In one embodiment, the microparticles are solid. In another embodiment, the microparticles are smooth or non-pitted. In another embodiment, the microparticles are homogenous or monolithic. In another embodiment, the microparticles have a morphology allowing for a high core load, high encapsulation efficiency, low burst, sustained release and syringeability.

Scanning electron micrograph images were obtained for microparticles of the present invention. External morphological examination of the microparticles indicates that the microparticles are smooth and non-pitted. Internal morphological examination of the microparticles indicates microparticles have a monolithic interior.

The microparticles of the invention may be administered to a patient in need of treatment by injection, nasal, pulmonary, oral, vaginal or other means of delivery. In one embodiment, the invention can be used to deliver a biologically active agent to any desired site, including, but not limited to, intramuscular, intradermal, subcutaneous, intraorbital, intraocular, intravitreal, intraaural, intratympanic, intrathecal, intracavitary, peritumoral, intratumoral, intraspinal, epidural, intracranial, and intracardial.

The microparticles may release a biologically active agent by any suitable means to allow for a controlled release of the biologically active agent. While not wishing to be bound by theory, the microparticles can release the biologically active agent by bulk erosion, diffusion or a combination of both. The microparticles are easily suspendable and syringable while able to provide increased duration, increased stability, decreased burst and controlled, sustained or delayed release of biologically active agents in vivo.

A surfactant is optionally used in order to provide formulations that have the required syringability. In one embodiment, a surfactant is used for providing a stable emulsion during the process of forming the microparticles of the present invention. In another embodiment, a surfactant is used for preventing agglomeration during drying during the process of forming the microparticles. In another embodiment, a surfactant is used for preventing agglomeration within the injection vehicle during the process of delivering the microparticles. Without wishing to be bound by theory, surfactants may provide batch-to-batch consistency of microparticles by forming a thin layer of material around the microparticles that helps prevent clumping. Any suitable surfactant may be used. Suitable surfactants include, but are not limited to, cationic, anionic, and nonionic compounds such as poly(vinyl alcohol), carboxymethyl cellulose (CMC), lecithin, gelatin, poly(vinyl pyrrolidone), polyoxyethylenesorbitan fatty acid ester (Tween 80, Tween 60, Tween 20), sodium dodecyl sulfate (SDS) and the like.

In one embodiment, the microparticles are formed using an emulsion comprising poly (vinyl alcohol). In a particular embodiment, the microparticles are formed using an emulsion comprising 1.0% poly (vinyl alcohol). The concentration of surfactant in the process medium is established to be an amount sufficient to stabilize the emulsion.

In one embodiment, the microparticles are lyophilized in a solution comprising SDS, Tween 20 or mannitol. In one particular embodiment, the microparticles are lyophilized in a solution comprising 7.8% SDS.

In one embodiment, the inherent viscosity of the biodegradable polymer may be in the range 0.1 to 2.0 dL/g. In another embodiment, the inherent viscosity of the biodegradable polymer ranges from about 0.1 to about 1.0 dL/g. In another embodiment, the inherent viscosity of the biodegradable polymer is about 0.15 dL/g, In another embodiment, the inherent viscosity of the biodegradable polymer is 0.3 dL/g. In another embodiment, the biodegradable polymer has an inherent viscosity of 0.6 dL/g. In another embodiment, the inherent viscosity of the biodegradable polymer is 0.9 dL/g.

Another embodiment of the invention provides a microparticle formulation comprising PEGylated insulin. The microparticles have a burst release in vivo and in vitro of less than 5%, a drug core load greater than 12% (w/w) and an encapsulation efficiency of greater than 80%. The microparticles have a mean diameter of less than 45 um, and greater than 90% (volume weighted) are between 25 and 63 um. The microparticles of this invention are capable of injection into a subject through needles of 25, 27 and 29 gauge or narrower, whereby the insulin plasma level is maintained for about one week to about four weeks.

Having generally described the invention, a further understanding can be obtained by reference to certain specific examples, which are provided herein for purpose of illustration only and are not intended to be limiting unless otherwise specified.

### EXAMPLES

The following examples are included to demonstrate particular embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation of Biodegradable Polymer Microspheres

A first phase containing 10% PLGA was prepared by dissolving 10 grams 85:15 PLGA (Medisorb 8515DLC01, Alkermes, Inc., 6960 Cornell Rd., Cincinnati, Ohio 45242) in 90 grams of ethyl acetate. The second phase was prepared by dissolving two grams of poly(vinyl-alcohol) (PVA) and 16 grams of ethyl acetate in 198 grams of water. Both solutions were placed inside separate temperature-controlled feed vessels at 20° C. (FIG. 2).

The second phase was pumped through a packed bed apparatus (6 mm Polytetrafluoroethylene (PTFE) tubing, 150 mm long, filled with 500μ glass beads) of the present invention at a rate of 1 ml/min. The first phase was pumped at the same time through the same packed bed apparatus at a flow rate of 1 ml/min. The emulsion was collected in an excess volume of water, the solvent was removed and the hardened microparticles separated.

The microspheres were analyzed by laser light scattering for size distribution with the following results:

Mean Diameter=46 μm (volume statistics)

D10=35 μm

D50=46 μm

D90=58 μm

### Example 2

### Preparation of 2-Methoxyestadiol (2ME) Microspheres

A first phase was prepared by dissolving 200 mg of 2ME and 400 mg of 50:50 PLGA in 7 ml of ethyl acetate. 2 grams of poly(vinyl-alcohol) (PVA) were dissolved in 198 grams of water to prepare the second phase. Both phases were then placed inside temperature-controlled water baths at 65° C.

The second phase was pumped through a packed bed apparatus (6 mm PTFE tubing, 150 mm long, filled with 500μ glass beads) of the present invention at a rate of 1.5 ml/min. The first phase was pumped at the same time through the same packed bed apparatus at a flow rate of 1 ml/min. The emulsion was collected inside a glass beaker where the solvent was removed from the emulsion droplets.

The hardened microspheres were centrifuged, and the microspheres were washed 3 times with water. The microspheres were analyzed for particle size distribution with the following results:

Mean Diameter=40 μm (volume statistics)

D10=27 μm

D50=40 μm

D90=53 μm

### Example 3

### Preparation of PEGylated Insulin Microspheres

A first phase was prepared by dissolving 213 mg of PEGylated insulin (PCT Publication WO 2004/010995 entitled “Method for Preparation of Site-Specific Protein Conjugates”) and 748 mg of 45:55 PLGA in 10 ml of methylene chloride. Next, 2 grams of poly(vinyl-alcohol) (PVA) were dissolved in 198 grams of water to prepare the second phase.

The first phase was pumped through a packed bed apparatus (6 mm PTFE tubing, 150 mm long, filled with 500μ glass beads) of the present invention at a rate of 1.7 ml/min. The second phase was pumped at the same time through the same packed bed apparatus at a flow rate of 0.7 ml/min. The emulsion was collected inside a glass beaker where the solvent was removed by evaporation.

The finished microspheres were filtered and washed with water, and then dried open to the atmosphere overnight. The dried microspheres were analyzed for particle size distribution with the following results:

Mean Diameter=61 μm (volume statistics)

D10=42 μm

D50=60 μm

D90=79 μm

### Example 4

### Preparation of Double-Emulsion Microspheres

A first phase was prepared by dissolving 4.5 g of 65:35 PLGA in 40.5 g ethyl acetate. Next, a second phase was prepared by dissolving 225 mg ovalbumin in 7.5 g water. Next, 2 g of poly(vinyl-alcohol) (PVA) and 5 g of ethyl acetate were dissolved in 192 g of water to prepare the third phase.

The first phase was pumped through the same packed bed apparatus at a flow rate of 5.0 ml/min. The second phase was pumped through a packed bed apparatus (1 inch stainless steel tube, 200 mm long, filled with 50μ glass beads) of the present invention at the same time at a rate of 1.0 ml/min. The internal emulsion coming out of the first packed bed apparatus as a result of the mixture of the first and second phases was then directed into a second packed bed apparatus (½ inch stainless steel tube, 200 mm long, filled with 500μ glass beads) of this invention. The third phase was pumped at the same time through the second packed bed apparatus at a rate of 13 ml/min. The resultant emulsion product coming out of the second packed bed apparatus was collected inside a glass beaker where the solvent was removed.

The finished microspheres were filtered and washed with water, and then lyophilized overnight. The dried microspheres were analyzed for particle size distribution with the following results:

Mean=35 μm (volume statistics)

Median=35 μm (volume statistics)

Standard Deviation=13.5 μm

### Example 5

### Preparation and Scale-Up of a Packed Bed Apparatus

An apparatus for the production of microparticles containing Estradiol Benzoate with a particle size distribution in the range of 25-60 microns was made of stainless steel tubing, 1 inch in diameter, and 200 mm in length. The tubing was packed with glass beads with an average diameter of 375 microns.

A 15% PLGA phase one solution was prepared by dissolving 150 grams 85:15 PLGA (Medisorb 8515DLC01, Alkermes, Inc., 6960 Cornell Rd., Cincinnati, Ohio 45242) in 850 grams of ethyl acetate. 75 grams of estradiol benzoate was added to the solution and stirred at 60° C. until completely dissolved. Next, 20 grams of poly(vinyl-alcohol) (PVA) and 100 grams of ethyl acetate were dissolved in 1880 grams of water to form the second phase. Both solutions were placed inside separate temperature-controlled feed vessels at 60° C. (FIG. 2).

The second phase was pumped through the above packed bed apparatus at a rate of 30 ml/min. The first phase was pumped at the same time through the same packed bed apparatus at a flow rate of 30 ml/min. The emulsion was collected inside a tank where the solvent was removed.

The finished microspheres were filtered and washed with water, and then dried under vacuum. The dried microspheres were analyzed for particle size distribution with the following results:

Mean=38 μm (volume statistics)

Median=38 μm (volume statistics)

Standard Deviation=8.4 μm

### Example 6

### Scale Up of Batch Size for Making Estradiol Benzoate Microspheres

In order to demonstrate the scalability of the Packed Bed apparatus and process, a 1 kg batch of microparticles containing Estradiol Benzoate were produced with a projected microparticle distribution range of 35-100 microns. A Packed Bed Apparatus was built of 1-inch stainless steel tubing, 200 mm in length and packed with glass beads having an average diameter of 500 microns.

A 16.7% PLGA first phase solution was prepared by dissolving 800 grams 85:15 PLGA (Medisorb 8515DLC01, Alkermes, Inc., 6960 Cornell Rd., Cincinnati, Ohio 45242) in 3990 grams of ethyl acetate. 200 grams of estradiol benzoate was added and stirred at 60° C. until completely dissolved. Next, 100 grams of poly(vinyl-alcohol) (PVA) and 500 grams of ethyl acetate were dissolved in 9400 grams of water to form the second phase. Both solutions were placed inside separate temperature-controlled holding tanks at 60° C. (FIG. 2).

The second phase was pumped through the packed bed apparatus at a rate of 50 ml/min. The first phase was pumped at the same time through the same packed bed apparatus at a flow rate of 50 ml/min. The emulsion was collected inside a tank where the solvent was removed.

The finished microspheres were filtered and washed with water, and then dried under vacuum. The dried microspheres were analyzed for particle size distribution with the following results:

Mean=66 μm (volume statistics)

Median=66 μm (volume statistics)

Standard Deviation=21 μm

### Example 7

### Scale Up of Packed Bed Apparatus and Flow Rates

This example demonstrates the application of a Packed Bed Apparatus for making microspheres at higher flow rates. A new Packed Bed Apparatus was built with stainless steel tubing 2-inch in diameter, 200 mm in length, and packed with glass beads with an average diameter of 465 microns.

A 10% PLGA first phase solution was prepared by dissolving 130 grams 85:15 PLGA (Medisorb 8515DLC01, Alkermes, Inc., 6960 Cornell Rd., Cincinnati, Ohio 45242) in 1170 grams of ethyl acetate. Next, 30 grams of poly(vinyl-alcohol) (PVA) and 210 grams of ethyl acetate were dissolved in 2760 grams of water to form the second phase. Both solutions were placed inside separate temperature-controlled feed vessels at 50° C. (FIG. 2).

The second phase was pumped through the above packed bed apparatus at a rate of 300 ml/min. The first phase was pumped at the same time through the same packed bed apparatus at a flow rate of 300 ml/min. The emulsion was collected inside a tank where the solvent was removed.

The finished microspheres were analyzed for particle size distribution with the following results:

Mean=28 μm (volume statistics)

Median=30 μm (volume statistics)

Standard Deviation=9.8 μm.

### Example 8

### Alternative Preparation of PEGylated Insulin Microparticles

PEGylated insulin was encapsulated in PLGA microspheres using the packed bed apparatus and method of Example 3 modified as follows. Insulin, specifically PEGylated at the PheB1 amino terminus (mono-methoxy PEG, molecular weight 4891 Da, propyl aldehyde reagent, NOF, Osaka, Japan) 3.75 gm, was co-dissolved with 11.28 gm PLGA (45:55 2A, Alkermes, Lebanon Ohio) in 100 mL methylene chloride. A water phase was prepared with 1% PVA. The water and organic phases were combined in a packed bed emulsifier of the present invention containing glass beads of 300-350 um diameter under laminar flow to produce an o/w emulsion. Solvent was evaporated from the emulsion droplets to form hardened microparticles encapsulating PEGylated insulin. The hardened microparticles had a mean diameter of 36.9 um before collection and drying. The microparticle suspension in water was passed through a 63 um screen and collected on a 25 um screen and dried, resulting in a particle size mean of 38.8 um and a distribution of >90% within the range 25 to 63 um. Drug content was 21% (w/w). The microspheres released 1.2% of the PEG-insulin after 3 hours in PBS, 37° C.

Scanning electron micrograph images of microparticles formed by the above process show that the microparticles have a smooth external morphology, and a monolithic internal morphology.

### Example 9

### Syringeability and Injectability of PEGylated Insulin Microparticle

350 mg of PEGylated insulin microparticles were suspended in 1.75 ml aqueous injection vehicle containing 0.5% CMC, 0.1% sodium dodecyl sulfate, 0.09% methylparaben (preservative) and 0.01% propylparaben (preservative). The microparticles suspended easily in the injection vehicle. The suspension was syringeable through a ½ in. long 27-gauge needle, (i.e. could be drawn up into a syringe and injected into air without blockage). A volume of ½ cc of the suspension was injected into chicken thighs subcutaneously using a 27 gauge needle. The injection was repeated several times and in each case the injections were successfully made without blockage.

Similar tests were conducted for PEG-insulin microspheres suspended in aqueous injection vehicle comprising CMC or hydroxypropylmethylcellulose (HPMC) without surfactant. Tests were done for microspheres with mean particle size (volume weighted) of 37.9 um at a concentration of 50 and 200 mg per ml vehicle. All samples suspended well and syringed well when drawn into syringes and then injected into air through a 27-gauge needle.

Syringeability tests were conducted for PEG-insulin microspheres suspended in aqueous injection vehicle comprising 0.5% CMC, 0.05% Tween-20, and PBS (10 mM Sodium Phosphate, 137 mM Sodium Chloride, and 2.7 mM Potassium Chloride, pH 7.4). Tests were done for microspheres with particle size between 25 and 63 um (mean 38.8 um) at a concentration of 600 mg in 1.0 ml injection vehicle. All samples suspended well and syringed well when drawn into syringes and then injected into air through a 27, 29, and 30 gauge needle.

### Example 10

Microparticles encapsulating PEGylated insulin made using the packed bed emulsifier were measured for their size distribution. Before sieving the particle size distribution had a mean diameter of 36.9 um with 12.2% of particle volume less than 25 microns, and 87.8% between 25 and 63 microns. Alternatively the pre-sieving distribution is described by d₁₀=17.9 um, d₅₀=39.7 um, and d₉₀=49.3 um. After sieving and drying, the particle size distribution had a mean diameter of 38.8 um with 99.9% of the particles between 25 and 63 um; d₁₀=30.8 um, d₅₀=38.5 um, and d₉₀=47.3 um.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

We claim:

1. A composition comprising:
a plurality of microparticles prepared by
(a) forming a first phase, comprising a solvent, one or more active agents and a polymer;
(b) forming a second phase comprising a solvent;
(c) passing said first phase and said second phase through a packed bed apparatus, resulting in the formation of microparticles; and
(d) collecting said microparticles containing said active agent;
wherein said microparticles, suspended in a diluent, are syringeable through a needle.

2. The composition of claim 1, wherein said packed bed apparatus comprises a packing material chosen from one or more of metal, ceramic, plastic, and glass.

3. The composition of claim 2, wherein said packing material comprises one or more of glass, stainless steel, or spherical beads.

4. The composition of claim 3, wherein said spherical beads range in size from about 20 to about 1000 microns in diameter.

5. The composition of claim 1, wherein >90% of said microparticles are from about 25 microns to about 63 microns in diameter.

6. The composition of claim 1, wherein the microparticles have a mean diameter of about 35-45 μm.

7. The composition of claim 1, wherein the microparticles have a diameter of less than or equal to 75% of the inner diameter of the needle.

8. The composition of claim 1, wherein the microparticles are capable of forming a free-flowing or un-agglomerated dry powder.

9. The composition of claim 1, wherein the microparticles are capable of forming a homogeneous or un-agglomerated suspension with a pharmaceutically acceptable, diluent, carrier, or excipient.

10. The composition of claim 9 wherein the pharmaceutically acceptable diluent, carrier, or excipient is an aqueous solution comprising one or more components selected from buffers, salts, non-ionic tonicity compounds, viscosity enhancers, stabilizers, antimicrobials, and surfactants.

11. The composition of claim 1, wherein the internal morphology of the microparticles is homogeneous or monolithic and the external morphology of the microparticles is smooth or non-pitted.

12. The composition of claim 1, wherein the microparticles, when suspended in an acceptable diluent, carrier or excipient at a concentration of from 50 to 600 mg/ml, are syringable through a needle chosen from one or more of a 25 gauge needle, a 27 gauge needle, a 29 gauge needle and a 30 gauge needle.

13. The composition of claim 1, wherein the microparticles are administered to a patient by intradermal, subcutaneous, intratumoral, intraocular or intramuscular injection.

14. A method of preparing microparticles, comprising:
(a) forming a first phase comprising an organic solvent, one or more active agents and a polymer;
(b) forming a second phase comprising an aqueous solvent;
(c) passing said first phase and said second phase through a packed bed apparatus under laminar flow conditions, resulting in the formation of microparticles; and
(d) collecting said microparticles containing said active agent;
wherein said microparticles, suspended in a diluent, are syringeable through a needle.

15. The method of claim 14, wherein 90% of said microparticles are from about 25 microns to about 63 microns in diameter.

16. The method of claim 14, wherein the microparticles have a mean diameter of about 35-45 μm.

17. The method of claim 14, wherein the microparticles are capable of forming a free-flowing or un-agglomerated dry powder.

18. The method of claim 14, wherein the microparticles are capable of forming a homogeneous or un-agglomerated suspension with a pharmaceutically acceptable diluent, carrier, or excipient.

19. The method of claim 14, wherein the pharmaceutically acceptable diluent, carrier or excipient is an aqueous solution comprising one or more components selected from buffers, salts, non-ionic tonicity compounds, viscosity enhancers, stabilizers, antimicrobials and surfactants.

20. The method of claim 14 wherein the internal morphology of the microparticles is homogeneous or monolithic and external morphology of the microparticles is smooth or non-pitted.

21. The method of claim 14, wherein the microparticles, when suspended in an acceptable diluent, carrier or excipient at a concentration of from 50 to 600 mg/ml, are syringable through a needle chosen from one or more of a 25 gauge needle, a 27 gauge needle, a 29 gauge needle and a 30 gauge needle.

22. The method of claim 14, wherein the microparticles are administered to a patient by intradermal, subcutaneous, intratumoral, intraocular or intramuscular injection.

23. A method of preparing microparticles comprising:
(a) forming a first phase comprising an organic solvent;
(b) forming a second phase comprising an aqueous solvent, one or more active agents and a polymer;
(c) passing said first phase and said second phase through a packed bed apparatus under laminar flow conditions, resulting in the formation of microparticles; and
(d) collecting said microparticles containing an active agent;
wherein said microparticles, suspended in a diluent, are syringable through a needle.

24. A method of preparing microparticles, comprising:
(a) forming a first phase comprising an organic solvent;
(b) forming a second phase comprising an organic solvent, one or more active agents and a polymer;
(c) passing said first and second phases through a packed bed apparatus under laminar flow conditions, resulting in the formation of microparticles; and
(d) collecting said microparticles containing an active agent;
wherein said microparticles, suspended in a diluent, are syringable through a needle.

25. A method of preparing microparticles, comprising:
(a) forming a first phase comprising an aqueous solvent, one or more active agents and an emulsion stabilizer;
(b) forming a second phase comprising an organic solvent and a polymer;
(c) forming a third phase comprising an aqueous solvent;
(d) passing said first and second phases through a packed bed apparatus to form a first emulsion;
(e) passing said first emulsion and said third phase through a second packed bed apparatus, resulting in the formation of microparticles;
collecting said microparticles containing said active agent;
wherein said microparticles, suspended in a diluent, are syringable through a needle.

26. An apparatus for the preparation of emulsion-based syringable microparticles containing one or more active agents, wherein the apparatus comprises a vessel and packing material situated therein.
